(19) United States
(12) Patent Application Publication
(10) Pub. No.: US 2018/0360739 A1
(43) Pub. Date: Dec. 20, 2018

(54) Non-Invasive Applications for Bioactive Agents

(71) Applicants: Celeste Joy Kirsten Lorenz, Scotts Valley, CA (US); Robert Paul Lorenz, Scotts Valley, CA (US)

(72) Inventors: Celeste Joy Kirsten Lorenz, Scotts Valley, CA (US); Robert Paul Lorenz, Scotts Valley, CA (US)

(21) Appl. No.: 15/625,384

(22) Filed: Jun. 16, 2017

(51) Int. Cl. A61K 9/00; A61K 47/22; A61K 45/06; A61K 36/185; A61K 47/46

(52) U.S. Cl. CPC A61K 9/0014; A61K 47/22; A61K 47/46; A61K 9/007; A61K 36/185; A61K 45/06

(57) ABSTRACT

The present invention relates to bioactive agents intended to act systemically, locally, and/or provide a sensory experience over an extended period of time. The non-invasive applications comprise one or more bioactive agent encapsulated in a carrier and crosslinked with a crosslinking agent. More specifically, applications can be self applied to the skin, hair follicles, mucosal membranes, and/or be inhaled by a mammal.

### FIELD OF THE INVENTION

The present invention relates to non-invasive applications for drugs, herbs, and/or other bioactive agents encapsulated within a carrier such as peptides, agar agar, and/or biologically acceptable organic moieties, and crosslinked with a crosslinking agent such as genipin (GNP) and/or proanthocyanidin (PA). More specifically, the application is prepared so that it can be transferred to the skin, hair follicles, or mucosal membrane of a mammal, and/or be inhaled by a person. Furthermore, the application is intended to provide a vitamin, health routine, drug, therapy, prevention, cure, and/or treatment that can be self applied to act locally, systemically, and/or provide the user with a sensory experience. After a predetermined amount of time, the bioactive agent(s), carrier(s), and crosslinking agent(s) are absorbed, distributed, biodegraded, dispersed, dissolved, diffused, eliminated, or any combination there of, from the application entry point and can be reapplied as desired.

### BACKGROUND OF THE INVENTION

The bioavailability of orally consumed drugs is limited by the gastrointestinal tract. Alternate administration routes, such as sublingual, inhalation, or rectal administration, do provide faster results, yet fail to produce long lasting effects. It is known that by non-invasive routes, such as transdermal absorbance, a substance can enter the bloodstream of a mammal, and exert its effects far from the original point of entry. Unfortunately, current treatments generally necessitate invasive methods such as intravenous or intramuscular injections to be reliable. Therefore, there is a need in the art for non-invasive drug delivery applications which can provide slow release of a drug or substance for a predetermined amount of time.

Two well known non-invasive transdermal drug release products in use today are the nicotine patch, and birth control patch. Negative factors that can result from use of these products include a social stigma associated with wearing a patch and having discomfort around the patch application area. Furthermore, patch's are generally designed to be used by adults for treating a limited number of medical conditions. Therefore, there is a need in the art to provide non-invasive drug delivery that can treat and/or prevent common conditions for a variety of ages with comfortable, discrete applications.

The majority of over the counter drugs, herbs, vitamins, and medications are only available as orally consumable pills or tablets. Some topical pain relief creams exist, but these are only designed to treat the area to which they are applied. Similarly, beauty products and cosmetics that have anti-aging claims work topically, and locally to the application point. Therefore, there is a need in the art to create a more readily bioavailable application for over the counter use designed to work locally, as well as systemically throughout the body for a predetermined amount of time.

The present invention intends to conduct further in vivo and in vitro testing to realize appropriate dosages, and optimal preparation. Certain descriptions, embodiments, and/or illustrations of the inventive composition contain forward-looking ideas, such as ideas concerning the anticipated outcome from use in a human subject, in vivo and in vitro test result predictions, future product performance, and other non-historical facts. The ideas and statements described herein are based on current expectations and currently available information. However, since these statements are based on factors that involve uncertainties, the inventive composition's actual performance, structure, and results may differ from those described and/or illustrated herein. There is no known product or patent currently claiming the present invention.

### BRIEF SUMMARY OF THE INVENTION

The inventive composition provides non-invasive applications comprising one or more bioactive agent encapsulated within a carrier that is crosslinked with a crosslinking agent.

In another aspect of the inventive composition the application is intended to provide a vitamin, herb, health routine, beauty product, drug, therapy, prevention, cure, remedy, alternative medicine, and/or treatment that can be self applied to act locally, systemically, and/or provide a sensory experience for an extended period of time.

In another aspect of the inventive composition the bioactive agent(s) is/are selected from a group comprising marine ingredients, minerals, botanicals, peptides, mucco-polysaccharides, polyphenols, trans-resveratrol, minerals, trace minerals, vitamins, trace elements, antioxidants, fruit acids, fungi, activated charcoal, diatomaceous earth, exogenous ketones, ketone esters, ketone salts, fermentations, distillations, probiotics, amino acids, amino acid salts, fatty acids, Co-Q10, omega-3, omega-6, proteins, essential oils, herbs, nutrients, whole foods, organic whole foods, sprouted foods, raw foods, superfoods, and/or other bioactive agents in the form of extracts, derivatives, and/or concentrates.

In another aspect of the inventive composition the bioactive agent(s) is/are encapsulated in a carrier from the group comprising peptides, agar agar, arginine, aloe vera, liposomes, polymers, disc and/or sphere shaped particles, nanoparticles, micro-particles, gold nanoparticles, silver nanoparticles, copper nanoparticles, multistage vectors, diatomaceous earth, moroccan red clay, pollen, and/or biologically acceptable organic moieties.

In another aspect of the inventive composition the crosslinking agent is genipin (GNP) and/or proanthocyanidin (PA).

In various embodiments the inventive composition optionally comprises one or more solvents, thickening agents, emulsifiers, waxes, oils, butters and/or additional additives.

In another aspect of the inventive composition drug-carrier-crosslinking agent compound is applied to the skin from under and/or within a drug delivery device such as a wearable product.

In another aspect of the inventive composition the application devise may be refillable.

In another aspect of the inventive composition the application devise may be disposable.

In another aspect of the inventive composition the drug-carrier crosslinked compound may be applied to skin or hair follicles of a mammal.

In another aspect of the inventive composition the drug-carrier crosslinked compound may be applied to mucosal membranes of a mammal.

In another aspect of the inventive composition the product may be inhaled into the lungs of a person.

In another aspect of the inventive composition the drug-carrier crosslinked compound is prepared for application as a health or beauty product in the form of a film, a foam, a cream, a lotion, a gel, a jelly, a wax, a butter, a moose, a spray, a mist, a scrub, a soak, a liquid, a salt, a tablet, a pill, a soap, a paste, a serum, an oil, a dye, an e-liquid, a vapor, a powder, a syrup, and/or a solid.

In the second embodiment of the inventive composition the application additionally comprises one or more body ink.

In the second embodiment of the inventive composition the drug-carrier-crosslinking agent-ink compound is applied in the form of a temporary tattoo.

In another aspect of the second embodiment the drug-carrier-crosslinking agent-ink compound is optionally applied in the form of a self tanning product.

### BRIEF DESCRIPTION OF DRAWINGS

While the invention has been described with respect to preferred embodiments, and illustrated as an example, it should be clear to those skilled in the art that modifications and improvements may be made without departing from the spirit and scope of the invention. Therefore, the invention is not to be limited by the specific illustrative embodiments, but only by the scope of the appended claims.

FIG. 1—FIG. 1 depicts a top view, side view, and sectional view, of an application film constructed according to various embodiments of the present invention.

FIG. 2—FIG. 2 illustrates a the points of preferred contact on a man according to various embodiments described herein.

FIG. 3—FIG. 3 shows the points of preferred contact on a woman according to various embodiments described herein.

FIG. 4—FIG. 4 depicts a person putting on and wearing an adjustable band according to various embodiments described herein.

FIG. 5—FIG. 5 illustrates a blown up view of an adjustable band according to various embodiments described herein.

FIG. 6—FIG. 6 shows a pregnant woman placing an application of the inventive composition on her abdomen according to various embodiments described herein.

FIG. 7—FIG. 7 illustrates an application suitable for a person to inhale.

### DETAILED DESCRIPTION OF PREFERRED EMBODIMENTS

The terminology used herein is for the purpose of describing particular embodiments only and is not intended to be limiting to the invention. As used herein, the term “and/or” includes any and all combinations of one or more of the associated items. As used herein, the singular forms “a,” “an,” and “the” are intended to include the plural forms as well as the singular forms, unless the context clearly indicates otherwise. It will be further understood that “comprises” and/or “comprising,” when used in this specification, specify the presence of stated bioactive agents, biological elements, non-active agents, molecules, compounds, and/or components, but do not preclude the presence or addition of one or more other bioactive agents, biological elements, non-active agents, molecules, compounds, components, and/or groups thereof. A “crosslinking agent” is intended to indicate a chemical agent that could crosslink two molecules. A “drug” and/or “bioactive agent” is/are meant to broadly refer to chemical molecules, biological molecules and/or bioactive agents providing a therapeutic, sensory, and/or prophylactic effect in vivo. A “drug” and/or “bioactive agent” may comprise, but is not limited to, synthetic chemicals, biotechnology-derived molecules, herbs, cells, genes, growth factors, health food, and/or alternate medicines. A “drug” and/or “bioactive agent” are referenced interchangeably according to specific components of the inventive composition.

Unless otherwise defined, all terms, including technical and scientific terms, used herein have the same meaning as commonly understood by one having ordinary skill in the art to which this invention belongs. It will further be understood that terms should be interpreted as having a meaning that is consistent with their meaning in the context of the relevant art and the present disclosure will not be interpreted in an idealized or overly formal sense unless expressly so defined herein.

In describing the invention, it will be understood that a number of bioactive agents are disclosed. Each of these has individual benefit and each can also be used in conjunction with one or more, or in some cases all, of the other disclosed bioactive agents. Let it be acknowledged that this includes a drug, herb, plant, and/or bioactive agent in it's listed form, and/or any and all elements, molecules, moieties, compounds, parts, fractions, portions, divisions, substrates, and/or products of all listed plants, herbs, drugs, and/or bioactive agents. It is further acknowledged that this optionally includes a derivative, cognate, extraction, fermentation, concentrate, infusion, concoction, dilution, solution, substance, diffusion, decoction, distillate, formula, elicitation, additive, constituent, potion, recipe, blend, mixture, mix, compound, suspension, tincture, emulsion, combination, culmination, substrate, product, and/or abstract of listed plants, herbs, drugs, and/or bioactive agents. Accordingly, for the sake of clarity, this description will refrain from repeating every possible combination of the individual bioactive agents, and non-active agents, in an unnecessary fashion. Nevertheless, the specification and claims should be read with the understanding that such combinations are entirely within the scope of the invention and the claims.

In the present invention, a bioactive agent has biological activity. Additionally, a biological carrier encapsulates one or more bioactive agents, controlling the rate of delivery and protecting against premature elimination. The carrier is crosslinked with a crosslinking agent selected from genipin (GNP) and/or proanthocyanidin (PA). Furthermore, a bioactive application optionally comprises a prodrug that is converted to the desired bioactive product in the skin, mucosal membranes, or layer thereof.

In another aspect of the present invention, the bioactive agent(s) is/are present at 0.0001% to 100%, including any level in between or any range therein. Preferably, a bioactive agent has a purity of 80% or greater. Ideally, a bioactive agent is 100% pure. An inventive composition is hypoallergenic. Optionally, all components of an inventive composition are plant based and/or herbal. Optionally, one or more components of an inventive composition are vegan, vegetarian, and/or food grade. Optionally, an inventive composition is non-toxic and considered safe if consumed. Furthermore, it is preferred an inventive composition be prepared in the most whole form suited for bioavailability and/or as a prodrug subsequently converted to a bioactive agent. Synthetically derived molecules will optionally be employed in an inventive composition. Optionally, synthetic components are predominantly absent from an inventive composition. Optionally, synthetic components are completely absent from an inventive composition. Optionally, an inventive composition is all natural. Optionally an inventive composition is certified organic. Optionally, an inventive composition comprises organic bioactive agent(s). Optionally, an inventive composition comprises wild harvested bioactive agents. It is acknowledged that an inventive composition may comprise 50% to 100% plant derived components. It is within the skill of the art to determine the optimal levels of individual components within an inventive composition in a concentrated solution and/or application.

In another aspect of the present invention, the bioactive agent(s) is/are provided in a carrier to protect bioactive agent(s) from degradation and premature elimination. The inventive composition comprises synthetic and/or organic carriers including but not limited to peptides, agar agar, arginine, aloe vera, liposomes, polymers, disc and/or sphere shaped particles, nanoparticles, micro-particles, gold nanoparticles, silver nanoparticles, copper nanoparticles, multistage vectors, diatomaceous earth, moroccan red clay, pollen, and/or biologically acceptable organic moieties. It is appreciated that an inventive composition optionally includes more than one carrier and that not all possible carries that could be used are listed herein. One of ordinary skill in the art will recognize that differing carriers will be operable herein depending on the application type of the inventive embodiment.

In another aspect of the present invention, the inventive composition is optionally formulated with levels of organic solvent. Optionally, less than 50% organic solvent is included. Optionally, an organic solvent is present at 5% or less. Optionally, an organic solvent is present at 2% or less. Optionally, an organic solvent is absent. Optionally, an organic solvent is water. Optionally, an organic solvent is buffered water. Optionally, an organic solvent is phosphate buffered water. Optionally, an organic solvent is saline. Optionally, an organic solvent is buffered saline. Optionally, an organic solvent is vinegar. Optionally, an organic solvent is acetic acid. An organic solvent is optionally an alcohol, such as isopropyl alcohol. It is appreciated that more than one organic solvent is optionally present in an inventive composition and that not all possible solvents that could be used are listed herein. It is further appreciated that the inventive composition be entirely solvent free.

In another aspect the inventive composition, a wax may act as a viscosity enhancer, emulsifier, emollient, and/or film forming agent. Optionally, the inventive composition comprises a wax. Optionally, the wax is beeswax, bayberry wax, candelilla wax, carnauba wax, and/or castor wax. Optionally the wax is beeswax. Optionally the wax is bayberry wax. Optionally the wax is candelilla wax. Optionally, a wax is absent. It is appreciated that an inventive composition optionally includes more than one wax and that not all possible waxes that could be used are listed herein. One of ordinary skill in the art will recognize that differing waxes, will be operable herein depending on the inventive embodiment.

In another aspect of various embodiments, the application's bioactive agents are prepared with a thickening agent. Optionally the thickening agent is present from 1% to 15%. Optionally the thickening agent is selected from a group consisting of natural gums and polysaccharides. Optionally the thickening agent is xanthan gum. It is appreciated that an inventive composition optionally includes more than one thickening agent and that not all possible thickening agents that could be used are listed herein. One of ordinary skill in the art will recognize that differing thickening agents, will be operable herein depending on the desired embodiment.

In another aspect of various embodiments, the inventive composition further comprises oil and/or butter. Optionally, the oil(s) and/or butter(s) is/are selected from the group consisting of oils and/or butters from coconut, jojoba, cocoa, almond, cashew, shea, sunflower, avocado, argan, flaxseed, and/or castor oil. Optionally the oil is coconut. Optionally the butter is shea butter. It is appreciated that an inventive composition optionally includes more than one oil and/or butter and that not all possible oils and/or butters that could be used are listed herein. One of ordinary skill in the art will recognize that differing oils and/or butters, will be operable herein depending on the desired consistency and/or embodiment.

The inventive composition optionally includes other additives, pharmaceutical carriers, stabilizers, surfactants, solvents, antioxidants, extracts, and/or emollients in the mixture or single component(s). The compositions of the invention are also optionally diluted to the appropriate level for application by using acceptable compounds and/or vehicles. The inventive composition optionally includes a scent and/or flavoring additive in addition to other elements described herein. A scent and/or flavoring may be natural and/or synthetic. It is preferred that any and all scent and/or flavoring additives be natural. One of ordinary skill in the art readily appreciates additives suitable for use with the present invention such as to provide desired flavor, scent, color, penetration characteristics, time release properties, and/or other desired characteristics.

It is preferred that a bioactive agent is derived from a plant, food, herb, fungus, botanical, and/or marine ingredient. The inventive composition, optionally comprises marine algae, botanicals, peptides, mucco-polysaccharides, polyphenols, trans-resveratrol, minerals, trace minerals, vitamins, trace elements, antioxidants, fruit acids, fungi, activated charcoal, diatomaceous earth, exogenous ketones, ketone esters, ketone salts, fermentations, distillations, probiotics, amino acids, amino acid salts, fatty acids, Co-Q10, omega-3, omega-6, proteins, essential oils, herbs, nutrients, whole foods, organic whole foods, sprouted foods, raw foods, superfoods, and/or other bioactive agents in the form of extracts, derivatives, and/or concentrates. It is acknowledged that bioactive agents may be in the form of a molecule, compound, and/or other biocompatible element. It is acknowledged that an inventive composition optionally comprises bioactive agent(s) and/or non-bioactive agents that are not listed herein. It is further acknowledged that (a) bioactive agent(s) may be used in more than one inventive composition. Furthermore, a bioactive agent may be used to treat, prevent, and/or cure a variety of conditions. One of ordinary skill in the art will recognize that a bioactive agent may be used to treat one or more condition. Additionally, it is acknowledged that all possible uses for a bioactive agent within an inventive composition, including conditions it could treat, prevent, and/or cure, may not be listed herein. One of ordinary skill in the art will recognize the ability for bioactive agents listed herein to treat or prevent a condition not listed herein without veering from the spirit or scope of the invention.

An inventive composition is optionally bioactive for up to 8 hours. Optionally, an inventive composition is bioactive for up to 12 hours. Optionally, an inventive composition is systemically bioactive for up to 24 hours. Optionally, an inventive composition is systemically bioactive for up to 48 hours. Optionally, an inventive composition is systemically bioactive for up to 3 days. Optionally, an inventive composition is systemically bioactive for up to one week. Optionally, an inventive composition is systemically bioactive for up to two weeks. Optionally, an inventive composition is systemically bioactive for up to one month. Optionally, an inventive composition is systemically bioactive for longer than one month and/or up to one year. Optionally, an inventive composition is systemically bioactive for one or more years.

It is appreciated that influencing moisture, temperature, oxidation, and pH levels can increase the ability of the application to penetrate the skin. Therefore, an inventive composition may additionally include a separately contained solvent, or solution. The solvent and/or solution may be provided in a dispensing container, a pre-moistened towelette, and/or a pre-saturated cloth. Optionally, a sterile, unsaturated, absorbent cloth is included with an application. The penetration enhancing solution is optionally saline, water, buffered saline, and/or buffered water and is optionally applied to skin prior to a body ink application. Optionally, the user is instructed to soak the sterile, absorbent cloth with warm water, and/or wash the skin with warm water before applying the solution. Optionally, the penetration enhancing solution is comprised within a layer of the application. The penetration enhancing solution is optionally the solvent when a solvent is present. Alternately the penetration enhancing solution is blended within an application. Optionally, a penetration enhancing solution is absent.

In another aspect of the inventive composition, the penetration enhancing component optionally comprises copper peptides, which promote production of glycosaminoglycans, like hyaluronic acid. Optionally, the penetration enhancing component comprises hydrolyzed rice proteins, glycine soja, and/or oxido reductases, which have the ability to improve oxygen supply to the tissue. Optionally, the penetration enhancing component comprises perfluoro-ester, which is a stabilized form of liquid oxygen that can act as a vehicle for other bioactive agents. Optionally, the penetration enhancing component comprises coconut shell based activated carbon. Optionally, the penetration enhancing component comprises agar agar, marine algae, and/or seaweeds. Optionally, the penetration enhancing component comprises magnesium. Optionally, the penetration enhancing component comprises methylsulfonylmethane (MSM). A penetration enhancing agent is optionally a buffering agent to achieve the desired pH optimal for penetration. A solution to enhance penetration is preferably buffered to a pH of 7-7.5. It is appreciated that an inventive composition optionally includes more than one penetration enhancing and/or buffering agent(s), and that not all possible penetration enhancing and/or buffering agents that could be used are listed herein. One of ordinary skill in the art will recognize that differing penetration enhancing agents, solvents, vehicles, and/or buffers will be operable herein depending on the type of embodiment.

Preferably an inventive composition is designed for over the counter use. Optionally, an inventive composition may require a prescription. Ingredients have been curated based on their demonstrated ability to act as a bioactive agent in treatments, therapies, cures, and/or preventative care for one or more condition, disease, symptom, and/or virus. It is acknowledged that ingredients intend to provide the user with a sensory experience, health care routine, therapy, treatment, cure, and/or prevention of a condition.

In another aspect of the inventive composition the bioactive agent(s) may be used to provide the user with a health boosting supplement such as, but not limited to a(n) immune system booster, hangover prevention, hangover treatment, pain reliever, sleep aid, weight loss supplement, sunscreen, full spectrum UV radiation protection, addiction recovery supplement, STD prevention, male contraceptive, female contraceptive, systemic insect repellent, mosquito repellent, tick repellent, flea repellent, sleep aid, vitamin, multi-vitamin, women's multi-vitamin, men's multivitamin, prenatal vitamin, breastfeeding vitamin, children's vitamin, infant vitamin, cognitive function booster, concentration enhancer, detoxifier, digestive aid, metabolism booster, energizing supplement, vitamin B complex, analgesic, anti-bacterial, antibiotic, probiotics, plant based recovery supplement, workout booster, brain enhancing supplement, study and concentration help, mental focus and memory supplement, heart health supplement, lung recovery supplement, plant calcium supplement, plant protein supplement, raw organic protein supplement, collagen builder, fermented food supplement, probiotic supplement, essential fatty acid supplement, Co-Q10 supplement, omega-3 supplement, omega-6 supplement, fatty acid supplement, enzyme supplement, mineral supplement, trace mineral supplement, amino acid supplement, antiviral, antioxidant complex, anti-inflammatory, polyphenol supplement, peptide supplement, organic raw food supplement, sprouted food supplement, exogenous ketone supplement, ketone esters, superfood supplement, herbal defense, virus protection, virus recovery, anti-fungal, anti-cholinergic, sexual enhancer, aphrodisiac, herbal mood enhancer, blood born illnesses protection, cancer prevention/treatment, heart disease prevention/treatment, Alzheimer's prevention/treatment, dementia prevention/treatment, high blood pressure prevention/treatment, high cholesterol prevention/treatment, cardiovascular disease prevention/treatment, diabetes prevention/treatment, migraine prevention/treatment, hair loss prevention/treatment, colic (babies) prevention/treatment, intestinal parasite prevention/treatment, ulcer prevention/treatment, arthritis prevention/treatment, acne prevention/treatment, eczema prevention/treatment, fungal infection prevention/treatment, energy supplement, depression prevention/treatment, dermatitis prevention/treatment, epileptic seizure prevention/treatment, gallbladder stone prevention/treatment, prostate protection, kidney stone prevention/treatment, hemorrhoid prevention/treatment, stretch mark prevention/treatment, cellulite prevention/treatment, varicose veins prevention/treatment, beautifying treatment/therapy, visible aging prevention and/or physical aging prevention, and/or combination thereof, and/or similar therapy, treatment, beauty routine, cosmetic product, preventative treatment, cure, remedy etc. It is appreciated by one skilled in the art that more than one stimulant, treatment, therapy, and/or preventative, may be provided by a supplement comprising bioactive agent(s) used according to the preferred embodiments listed herein. It is further appreciated that bioactive agent(s) may be combined with one or more other bioactive agent(s). It is further acknowledged, and obvious to one skilled in the art, that an inventive composition may be used to treat, cure, and/or prevent a condition, virus, disease, and/or illness and/or provide the user with an experience, that is not listed herein, without veering from the spirit or scope of the invention.

In various aspects of the present invention, the bioactive agent(s) may broadly comprise, but are not limited to, synthetic chemicals, biotechnology-derived molecules, herbs, health food, extracts, and/or alternative medicines. Furthermore, bioactive agents may optionally comprise analgesics/antipyretics, antiasthmatics, antibiotics, antidepressants, antidiabetics, antifungal agents, antihypertensive agents, anti-inflammatories, antineoplastics, antianxiety agents, immunosuppressive agents, antimigraine agents, sedatives/hypnotics, antianginal agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterial agents, antiviral agents, anti-infectives, bronchodilators, steroidal compounds and hormones, hypoglycemic agents, hypolipidemic agents, proteins, nucleic acids, agents useful for erythropoiesis stimulation, antiulcer/antireflux agents, antinauseants/antiemetics, as well as other drugs useful in the compositions and methods described herein.

In one aspect of the present invention the bioactive agent(s) is/are selected from a group of ingredients extracted from marine algae and/or botanicals comprising of: brown algae polyphenols, fucose, laminaran, mucco-polysaccharides, plankton, red algae, brown algae, marine algae, thiotaine, resveratrol, phytosphingolipids, gamma-linolenic acid (GLA), calcium, magnesium, potassium, copper, silica, maganese, iodine, zinc, arbutin, dictyopteris membranacea, retinol, tetrahexydecyl ascorbate, trans-resveratrol, copper peptides, dipeptide diaminobutroly benzylamide diacetates, hydrolyzed rice proteins, glycine soja, oxido reductases, caprylic capric triglycerides, hexapeptide-11, octapeptide-3, perfluoro-ester, laminaria extract, micrococcus lysate, 8-oxo-guanine glycosylase, orphidium plankton, solar photospheres, white tea extract, vitamin C ascorbic acid, vitamin C ester, vitamin E, magnesium ascorbyl phosphate (MAP), ferulic acid, brown algae polyphenols, beta-carotenes, aminoguanidine HCL, anthocyanins, and/or parts, derivatives, cognates, and/or mixtures thereof. Bioactive agents selected from this group of marine ingredients and botanicals may be employed in anti-aging treatments, UV radiation protection, DNA repair, free radical scavenging, cell protection, skin damage reversal, internal health, and/or beauty treatments. It is appreciated that an inventive composition optionally includes more than one bioactive agent and that not all possible bioactive agents that could be used are listed herein. One of ordinary skill in the art will recognize that differing bioactive agent(s), will be operable herein depending on the desired effect, application type, and/or embodiment.

In another aspect the inventive composition the bioactive agent(s) act as UV protection, and/or UV damage repair. Optionally bioactive agent(s) are selected from a group comprising prosopis juliflora, cercidium floridum, cercidium microphyllum, and parkinsonia aculeata. Optionally, (a) bioactive agent(s) may be pollen from a plant in the group comprising mesquite, prosopis juliflora, mexican palo verde, parkinsonia aculeata, blue palo verde, cercidium floridum, foothills palo verde, and/or cercidium microphyllum. Furthermore, (a) bioactive agent(s) are optionally selected from a group of plant extracts comprising parts of selaginella, magnolia, snow lotus, and/or jasmine. Furthermore, (a) bioactive agent(s) are optionally selected from a group of peony clever oil, ylang ylang pure dew, rose hydrosol, trehalose, hyaluronic acid, and/or combinations thereof. Optionally, an inventive composition further comprises methoxy cinnamic acid ethylhexyl, alkyl benzoate, salicylic acid ethylhexyl, myristoyl Qu polyglycerol ester, cyclic polydimethylsiloxane, diethylamino hydroxyl benzoyl hexyl benzoate, methylbenzyl subunit camphor, european plum fruit extract and/or combinations thereof. Optionally, an inventive composition further comprises purslane and/or skullcap extract. Optionally, an inventive composition further comprises aloe vera. It is further appreciated that a UV protection, UV repair, and/or sunscreen comprises bioactive agents that work systemically. Optionally, an inventive composition for UV protection and/or UV repair, include(s) more than one bioactive agent(s) and that not all possible bioactive agents that could be used are listed herein. One of ordinary skill in the art will recognize that differing bioactive agent(s), will be operable herein depending on the application type and/or embodiment. Furthermore, bioactive agents listed above may be used in treatments, therapies, supplements, and/or compositions other than the listed intent. It is acknowledged that bioactive agents listed herein may be used to treat or prevent a variety of conditions.

In another aspect of the inventive composition the bioactive agent(s) may be used to provide the user with a sensory experience. Optionally the bioactive agent(s) intended to provide a sensory experience are selected from the group of cannabis extracts comprising, tetrahydrocannabinol (THC), cannabinol (CBN), cannabidiol (CBD), cannabigerol (CBG), cannabidivarin (CBDV), and cannabichromene (CBC), and/or any combination thereof. Optionally, a cannabis extract is the bioactive agent. Optionally tetrahydrocannabinol (THC) is the bioactive agent. It is appreciated by one skilled in the art that more than one bioactive agent may be included to provide a sensory experience. It is further acknowledged that bioactive agent(s), including tetrahydrocannabinol (THC), cannabinol (CBN), cannabidiol (CBD), cannabigerol (CBG), cannabidivarin (CBDV), and cannabichromene (CBC) may have purposes beyond that of a sensory experience including a treatment, prevention, therapy, remedy, and/or cure for one or more conditions such as seizures, eating disorders, insomnia, chronic pain, nausea, etc. that may be employed by the inventive composition. It is appreciated that an inventive composition optionally includes more than one bioactive agent and that not all possible bioactive agents that could be used are listed herein. One of ordinary skill in the art will recognize that differing bioactive agent(s), will be operable herein depending on the desired effect, application type, and/or embodiment.

An inventive composition designed to prevent pregnancy is achieved with an drug-carrier-crosslinking agent compound applied to the skin, mucosal membranes, and/or inhaled by a person. To increase initial delivery speed, an inventive composition is optionally formulated to dissolve when placed in contact with mucosa, such as that in the vulva, anus, and/or mouth. Optionally, bioactive agent(s) is/are selected from a group comprising lignosulfonic acid (LSA), polyanetholesulfonic acid (PASA), polyvinylsulfonic acid, and/or other sulfonic acid derivatives and/or combinations thereof. Optionally, bioactive agent(s) comprise hydroalcoholic extract of annona squamosa. Optionally, bioactive agent(s) comprise unripe papaya seed extract and/or concentrate. Optionally the bioactive agent(s) act as a male contraceptive. Optionally the bioactive agent(s) act as a female contraceptive. Optionally the bioactive agent(s) act as a natural contraceptive. Optionally the bioactive agent(s) act as protection against a sexually transmitted disease. An inventive composition is optionally designed to prevent the transmission of disease through blood or bodily fluids. An inventive composition is optionally prepared for the prevention and/or treatment of herpes, genital human papillomavirus (HPV), chlamydia, trichomoniasis, gonorrhea, genital herpes, syphilis, and human immunodeficiency virus (HIV). Optionally, (a) bioactive agent(s) intended to protect from and/or treat a blood born disease or illness including STDs is/are selected from a group comprising garlic, tea tree, milk thistle, gentian root, lycopene, and/or berberine. It is appreciated that an inventive composition to treat or prevent STDs optionally includes more than one bioactive agent and that not all possible bioactive agents that could be used are listed herein. Optionally, the bioactive agent(s) act as protection against HIV. Optionally, the bioactive agent(s) work to decrease sperm production, implantation, and/or prevent pregnancy in a mammal by influencing hormones. It is acknowledged that the bioactive agent(s) can optionally be anti-bacterial, anti-viral, anti-microbial, provide immune defense, and/or act as a natural antibiotic and/or probiotic. It is further appreciated that an inventive composition that acts as a contraceptive optionally includes more than one bioactive agent and that not all possible bioactive agents that could be used are listed herein.

In another aspect of the inventive composition, the bioactive agents are designed to increase fertility in a male or female. Natural remedies to increase fertility may include active agents listed herein including, but not limited to, marine ingredients, herbal remedies, and/or plant extracts. However, it is acknowledged that fertility may increase due to other factors, such as overall health and well being. Often emotional calm and relaxation can increase chances of conception. Therefore a fertility treatment may comprise vitamins and herbs, as well as other calming and health boosting supplements.

In another aspect the inventive composition the bioactive agent(s) act as a detoxifier. Optionally, the detoxifier is intended to help rid the body from chemical build up such as can be obtained from exposure to pesticides, synthetic topical cosmetics, BPA products, PCBs, consumer products, industrial pollutants, flame retardants, food packaging, environmental waste, DDT, and household products comprising endocrine-disrupting chemicals (EDC). Optionally, (a) bioactive agent(s) is/are selected from a group comprising chlorella, chlorophyll, blue green algae, brown algae, seaweed, spirulina, alfalfa, dulse, chard, arugula, wheatgrass, watercress, asparagus, beets, blueberries, kale, spinach, collard greens, sweet potatoes, tomatoes, citrus fruit, garlic, berberine, green tea, white tea, mustard seed, broccoli sprouts, cabbage, cranberries, fennel, goji berries, mung beans, coconut oil, hemp oil, olive oil, avocado oil, flaxseed oil, almond oil, pineapple enzymes, pumpkin seed, chia seed, sunflower seed, sesame seed, black current seed, siberian cedar nuts, brazil nuts, psyllium seed, triphala fruit extract, marshmallow root, ginger rhizome, cinnamon bark, dandelion, turmeric, red clover, stinging nettle, burdock root, cilantro, chives, basil, sage, bay leaf, rosemary, thyme, parsley, ground ivy, milk thistle, neem, wormwood, black walnut hull, american wormseed, and hibiscus. It is appreciated that an inventive composition to reduce internal toxic buildup works systemically. It is further appreciated that an inventive composition to reduce toxic buildup includes more than one bioactive agent, and that not all possible bioactive agents that could be used are listed herein. One of ordinary skill in the art will recognize that differing bioactive agent(s), will be operable herein depending on the application type and/or embodiment. Furthermore, bioactive agents listed above may be used in treatments, therapies, supplements, and/or compositions other than the listed intent. It is acknowledged that bioactive agents listed herein may be used to treat or prevent a variety of conditions.

In another aspect the inventive composition the bioactive agent(s) act as insect repellent. Optionally, (a) bioactive agent(s) is/are selected from a group comprising essential oils such as para-menthane doil (PMD), geraniol, lemongrass oil, garlic oil, cinnamon oil, cumin oil, camphor oil, turmeric oil, peppermint oil, lemon eucalyptus oil, eucalyptus oil, tea tree oil, rose geranium oil, rosemary oil, lavender oil, thyme oil, neem oil, azadirachtin, pyrethrum, pyrethrin, citronella, citronella java, cedarwood oil, valerian oil, litsea cubeba oil, and/or general citrus oils. Optionally the inventive composition further comprises a bio-chemical selected from the group consisting of meliantriol, nimbinin, nimbidin, nimbidol, thionimone, nimatone, nimidol, nimbedic acid, nimbedinic acid, neo-nimbidin, nimidinin, gedunim, vapinin, meldenin, and/or vilasinin. Optionally the inventive composition further comprises a bio-chemical selected from the group consisting of azadirone, azadiradione, beta hydroxy azadiradione, and/or delta epi azadiradione. Optionally the inventive composition further comprises a bio-chemical selected from the group consisting of beta sitosterol, kaemferol, quercetin, and/or myricetin. Optionally, the inventive composition comprises octanoic acid, nonanoic acid, and/or decanoic acid. Optionally the inventive composition is virtually sent free. Optionally, the inventive composition comprises diatomaceous earth. Optionally, the inventive composition comprises oleic acid. Optionally, the inventive composition comprises algae. Optionally, the inventive composition comprises aloe vera. Optionally, the inventive composition comprises black cumin seed oil. Optionally, the inventive composition comprises litsea cubeba oil, garlic oil, and oleic acid. Furthermore, it is acknowledged that an inventive composition intended to act as an insect repellent may work effectively against mosquitoes, fleas, chiggers, spiders, ticks (including deer ticks), mites, bees, yellow jackets, gnats, beetles, horse flies, and the biting midge, etc. Furthermore, it is appreciated that an inventive composition is DEET free. It is appreciated that an inventive composition to repel insects works systemically. It is further appreciated that an inventive composition to repel insects optionally includes more than one repelling agent and that not all possible repelling agents that could be used are listed herein. One of ordinary skill in the art will recognize that differing repelling agent(s), will be operable herein depending on the application type and/or embodiment.

An inventive composition to prevent fleas, ticks, and mites is optionally formulated for use on an animal. Optionally, an inventive composition prevents tick bites, mites, and flea infestations in dogs and cats. Optionally an inventive composition prevents fly bites, mites, and ticks on farm animals. An inventive composition suited for use on an animal may protect animals from mosquitoes, fleas, chiggers, spiders, ticks (including deer ticks), mites, bees, yellow jackets, gnats, beetles, horse flies, and the biting midge. Additionally, an inventive composition prepared for use on an animal may prevent heart worm, ring worm, tapeworm, roundworm, hookworm, lyme disease, hot spots, skin irritations, and/or ear infections.

In another aspect the inventive composition the bioactive agent(s) are intended to treat and/or prevent obesity. Optionally, an inventive composition may include bioactive agent(s) that can improve metabolism, decrease food cravings, eliminate fat buildup, increase energy, increase nutrient absorption, improve digestion, balance hormones, reduce bad cholesterol, build muscle, improve muscle tone, maximize a workout, aid recovery from a workout, and other claims that relate to loosing weight, building muscle, overall fitness, getting in shape, and/or toning the body. Optionally (a) bioactive agent(s) is/are selected from a group comprising exogenous ketones, ketone salts, and ketone esters including acetone, acetoacetic acid, beta-hydroxybutyric acid (BHB), beta-hydroxybutyrate mineral salt (BMS), medium chain triglyceride oil, ketone ester-1,3 butanediol acetoacetate diester (KE), 1,3-butanediol (BD), and/or derivatives thereof. Furthermore, an inventive composition may comprise bioactive agents from a group of known weight control agents comprising chlorogenic acid, garcinia cambogia, glucomannan, sphaeranthus indicus, garcinia mangostana, conjugated linoleum acid (CLA), forskolin, caffeine, chlorella, berberine, and/or mixtures thereof. It is further appreciated that an inventive composition to reduce obesity optionally includes more than one bioactive agent and that not all possible bioactive agents that could be used are listed herein. It is acknowledged the bioactive agent(s) including exogenous ketones, ketone salts, and ketone esters may have purposes beyond that of weight loss including a treatment, prevention, therapy, remedy, and/or cure for one or more conditions. One of ordinary skill in the art will recognize that differing bioactive agent(s), will be operable herein depending on the application type and/or embodiment.

In another aspect of the inventive composition, the bioactive agent(s) is intended to treat and/or prevent a hangover. Bioactive agent(s) for treating and/or preventing a hangover are selected from a group that comprises activated charcoal, ginger, chlorella, berberine, royal jelly, magnesium, zinc citrate, niacin, folic acid, potassium, quercetin, L-Glutathione, cysteine, N-Acetyl cysteine, glutamic acid, glycine, vitamin B1, vitamin B2, vitamin B5, vitamin B6, vitamin B12, vitamin C, vitamin D, honey, aloe vera, prickly pear, coconut, milk thistle, artichoke, sarsaparilla, spirulina, goji berry, acai, oyster extract, taurine, chitosan, seaweed, algae, agar agar, and/or derivatives or combinations thereof. It is appreciated that an inventive composition to treat or prevent a hangover optionally includes more than one bioactive agent and that not all possible bioactive agents that could be used are listed herein.

In another aspect of the inventive composition, the bioactive agent(s) is/are intended to provide pain relief. Bioactive agent(s) for treating and/or preventing pain are selected from a group that comprises dl phenylalanine, curcumin, curcuminoids, boswellia, ginger, turmeric, DLPA, L-tyrosine, L-phenylalanine, L-tryptophan, L-threonine, L-leucine, L-lysine, nattokinase and/or derivatives or combinations thereof. Optionally, bioactive agents for treating or preventing pain are selected from the group of cannabis extracts comprising, tetrahydrocannabinol (THC), cannabinol (CBN), cannabidiol (CBD), cannabigerol (CBG), cannabidivarin (CBDV), and cannabichromene (CBC), and/or any combination thereof. It is appreciated that an inventive composition to provide pain relief optionally includes more than one bioactive agent and that not all possible bioactive agents that could be used are listed herein.

In another aspect of the inventive composition, the bioactive agent(s) is/are intended to provide mental performance, brain growth, and cognitive enhancement. Furthermore, an inventive composition is optionally designed to treat depression, treat mental illness, and/or aid in addiction recovery. An additional function of an inventive composition includes bioactive agent(s) effective in treating Alzheimer's disease and dementia. Bioactive agent(s) for the inventive composition designed for providing improved mental performance are selected from a group that comprises St. John's wort, S-adenosylmethionine (SAMe), hydroxytryptophan (5-HTP), DPLA, L-tyrosine, L-phenylalanine, L-tryptophan, L-threonine, L-leucine, L-lysine, ash tree bark, phellodendron amurense (amur cork tree bark), ashwaganda, passion flower, bacopa, and/or derivatives or combinations thereof. Optionally (a) bioactive agent(s) is/are selected from a group comprising nootropic compounds including N-phenylacetyl-L-prolylglycine ethyl ester, hurperzine A, phenylethylamine (PEA), uridine monophosphate, phosphatidylserine, hordenine HCL, vinpocetine, theobromine, DHEA, pterostilbene, and caffeine. Additionally, an inventive composition comprises bioactive ingredients from a group of choline donors including centrophenoxine, citicoline (CDP choline), and alpha GPC. Optionally an inventive composition will further comprise bioactive agent(s) selected from a group of amino acids including acetyl-L-carnitine, N-acetyl tyrosine, DL-phenylalanine, taurine, and L-theanine. Optionally, bioactive agents are additionally selected from the group of neuro-vitamins comprising benfotiamine, niacinamide, calcium pantothenate (P-5-P), methylcobalamin, ascorbic acid, and microencapsulated cholecalciferol. Optionally, bioactive agents are additionally selected from the group of adaptogen extracts comprising bacopa monnieri, mucuna pruriens, ginkgo biloba, coleus forskohlii, artichoke extract, rhodiola rosea, lion's mane, and gynostemma pentaphyllum. Optionally, bioactive agents are additionally selected from the group of neuro-minerals comprising lithium orotate, magnesium threonate, and zinc picolinate. Optionally, bioactive agents are additionally selected from the group of neuro-anti-inflammatories and antioxidants comprising bioPQQ, quercetin, curcumin, algal DHA, green tea extract, and bioperine. Optionally an inventive composition additionally comprises caffeine. Optionally an inventive composition additionally comprises chlorella. It is appreciated that an inventive composition optionally includes more than one bioactive agent and that not all possible bioactive agents that could be used are listed herein.

An inventive composition provides controlled drug delivery to the bloodstream of a mammal. Optionally, an inventive composition will be deemed safe for all ages. Optionally, an inventive composition is designed for use by people 18 and older. Optionally, an inventive composition is designed for use by people 21 and older. Optionally, an inventive composition is designed for use by people 60 and older. Optionally, an inventive composition is designed for use by infants. Optionally, an inventive composition is designed for use by children 2 and under. Optionally, an inventive composition is designed for use by children 12 and under. Optionally, an inventive composition is designed for use by teens. Optionally, an inventive composition is designed for use by women. Optionally, an inventive composition is designed for use by pregnant women. Optionally, an inventive composition is designed for use by lactating women. Optionally, an inventive composition is designed for use by men. Optionally, an inventive composition is designed for use by an animal. Optionally, an inventive composition is designed for use by a dog or cat. Optionally, an inventive composition is designed for use by a farm animal.

In another aspect of the inventive composition applications may be prepared for cosmetic purposes. An inventive composition is optionally prepared as a lotion, cream, powder, gel, scrub, bath soak, exfoliant, body wash, toothpaste, dry shampoo, leave in conditioner, hair dye, hair styling product, deodorant, and/or make-up. Optionally, the composition of bioactive agents will dissolve in warm water, such as in a bath soak. Optionally, the composition of bioactive agent(s) are applied to the hair or scalp, such as in a dry shampoo, leave in conditioner, dye, and/or styling product. Optionally, the composition of bioactive agent(s) are applied to the armpit, such as in a deodorant application. Optionally, the composition of bioactive agent(s) are applied to the teeth such as in a toothpaste, chewable tablet, film strip, or gum gel. Optionally, the composition of bioactive agent(s) are applied to the skin and worn under an occlusion device, such as a neoprene wrap, to induce heat accelerated absorption. For example a cream preparation to induce weight loss may be applied to the abdomen and covered with a body wrap.

In the first embodiment of the inventive composition the crosslinking agent is proanthocyanidin (PA) and the drug-carrier-crosslinking agent application is prepared as a film or foam. The film or foam is affixed to a semi-firm, flexible backing when prepared for use within a delivery devise. The application prepared in the form of a film or foam with a backing can be inserted into a refillable drug delivery devise. In this embodiment, the devise is designed for a refillable application to be inserted into a slot that holds the application in contact with the skin of a mammal for the duration of drug delivery.

In a similar preparation the application, including the bioactive agent(s), is/are contained within a disposable product and/or devise such as a bracelet, wristband, wrap, tie, impregnated tampon, condom, vaginal ring, suppository, penile band, sponge, contact lens, inhaler, cigarette, e-cigarette, a cosmetic product and/or other application intended to be discarded after use.

In another aspect of the inventive composition, an inventive composition is prepared to be inhaled into the lungs of a person and/or applied in contact with mucosal membranes. The drug-carrier-crosslinked compound is applied or inhaled with a product selected from a group comprising a film, a foam, a contact lens, an eye rinse, an impregnated tampon, a douche, an enema, a semi-permanent tattoo, a condom, a suppository, a vaginal ring, a tablet, a pill, a vaporizer, a humidifier, an e-cigarette, an inhaler, and/or a dissolvable application. It is acknowledged that an applicator may be included with an inventive composition. It is further acknowledged that an inventive composition prepared for a mucosal application may be placed in the mouth, under the tongue, inside the check, inside the vulva, inside the anus, in the eye, and/or inhaled into the lungs of a mammal.

In a second embodiment the inventive composition additionally comprises a concentrated body ink. The inventive composition optionally comprises genipin, henna, woad, madder, achiote, indigo, gold, copper, and/or silver, alone, or in addition, to a natural plant, fruit, flower, and/or vegetable, dye, ink, pigment, extraction, reduction, and/or concentration such as from one or more in the group comprising onion, blueberries, coffee, mango, papaya, cranberry, chlorophyll, beetroot, barberry, raspberry, paprika, saffron, safflower, birch, tea, bay leaves, turmeric, carrot, cocoa, nettles, goldenrod, lily of the valley, hibiscus, roses, cherries, walnut, black walnut, espresso, dandelion, oak bark, sunflower, st. John's wart, and/or citrus dyes. Optionally, dihydroxyacetone (DHA) is employed in an inventive composition as a skin staining agent. Optionally, gold, silver, and/or copper are visually present in an inventive composition. An inventive composition intended for temporary use optionally washes off with soap and water, a cleansing solution, an oil, and/or gentle exfoliation. It is appreciated that more than one body ink is optionally present in an inventive composition and that not all possible plant dyes or body inks that could be used are listed herein.

In the second embodiment genipin may act as the crosslinking agent as well as the ink component. The application may be prepared in the form of body ink or a temporary tattoo. The application process resembles that of applying a temporary tattoo. In prior art genipin has been used in body ink compositions and as a crosslinking agent. Concentrated genipin is capable of staining the epidermal layers for approximately two weeks. The ink component may further act as a drug delivery vehicle. Optionally, the application film can be removed after the bioactive components have transferred into the skin and/or mucosal membranes. Optionally, the film application dissolves after transfer to mucosal membranes. Typically, the application film remains in place after application for approximately 10 to 15 minutes. Optionally, the application film remains in place for up to 20 minutes. Optionally, the application film remains in place after application for up to 30 minutes. Optionally, the application film remains in place after application for up to 45 minutes. Optionally, the application film remains in place after application for up to one hour. Optionally, the application film remains in place after application for up to three hours. Optionally, the application film remains in place after application for up to six hours. Optionally, the application film remains in place after application for up to eight hours. Optionally, the application film remains in place after application for 24-48 hours. Optionally, the application film remains in place for the entire duration of use. Optionally, the application film peels off. Optionally, the application film requires oil to be removed. Optionally, the application film requires exfoliation to be removed.

In body ink applications the ink transfers to the layers of skin or mucosal membrane. The ink preferred for body ink compositions is genipin. Genipin is found in the fruit of the Gardenia (gardenia jasminoides ellis), among other plants, and will change from clear to dark blue-black when in contact with amino acids. Different amino acids can give genipin a variety of blue-black colors. Other methods for preparation can produce a red-orange color from genipin. Furthermore, genipin is known to act as a crosslinking agent, which can slow biodegradation, while remaining porous. Genipin concentrate used for body ink compositions in the present invention comprise genipin with 90% or greater purity. Genipin concentrate may be dissolved in a solvent, such as water, saline, buffered water and/or buffered saline.

In another aspect of the inventive composition when an ink component is present, the visual disappearance of body ink, such as by bioabsorbtion, bioerosion, diffusion, elimination, biodegradation and/or a combination thereof, serves as a means to visually prompt the user when it is time to reapply. More specifically, the amount of bioactive agent within a carrier corresponds to the amount of ink present within a composition so that the application remains bioactive, at the appropriate dosage level, for the same duration of time that the body ink is visible.

Optionally henna is the ink component. The dying agent in henna is lawsone, which is know to attach it's self to proteins. Optionally henna is combined with another ink, pigment, and/or dye. Optionally henna and genipin are used jointly in an inventive composition. Optionally henna and dihydroxyacetone (DHA) are used jointly in an inventive composition. Optionally the ink component is available in a variety of skin colors. It is acknowledged that henna can be used as the ink component, a bioactive agent, and/or a drug delivery vehicle.

In the second embodiment the inventive composition is optionally applied as a self tanning product. A self tanning product includes the drug-carrier-crosslinked compound as well as one or more body inks. A self tanning product optionally comes in a variety of skin tones. The self tanning product may include genipin in its red form. A self tanning product optionally comprises dihydroxyacetone (DHA). Dihydroxyacetone (DHA), may be additionally employed as a skin tinting agent in an inventive composition. A self tanning product optionally comprises henna. Henna is optionally the ink component in a self tanning product. A self tanning product may additionally comprise herb and/or plant dyes such as those that could be blended to match skin tones appearing tan including but not limited to mango, papaya, onion skins, coffee, cocoa, coconut, walnut, black walnut, espresso, carrot, tea, dandelion, avocado, safflower, St. Johns wart, turmeric, barberry, beetroot, goldenrod, burdock, and/or bee pollen. An ink may additionally act as a drug-carrier-crosslinking agent/vehicle. It is appreciated that a self tanning product optionally comprises more than one type of body ink and that not all possible inks, dyes, or pigments that could be used are listed herein. One of ordinary skill in the art will recognize that differing inks, dyes or pigments will be operable herein depending on the desired effect, application type, color tone, and/or embodiment. It is acknowledged that additional plant extracts and concentrates may be employed in an inventive composition without veering from the spirit or scope of the invention.

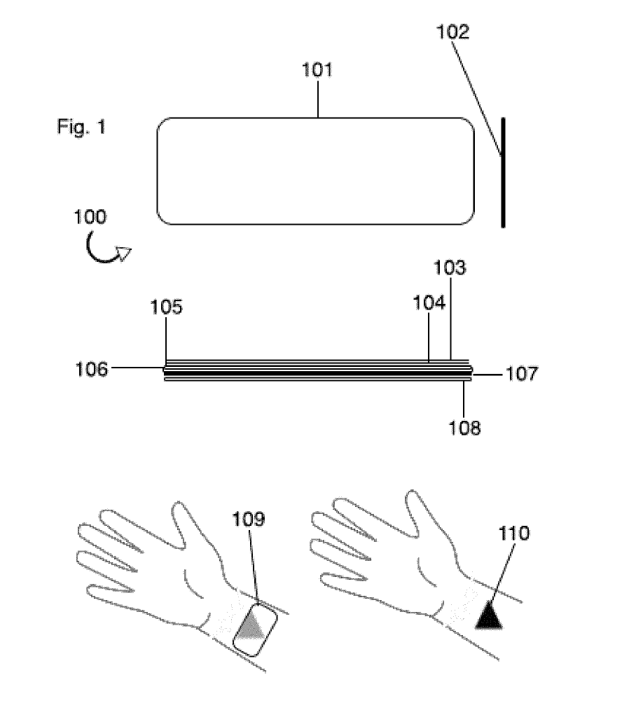

The inventive composition, illustrated in FIG. 1 depicts a blown up sectional view of an application film constructed according to the second embodiment of the present invention. The film is shown from a top view in it's transparent form before being applied (101). The side angle view (102) depicts the final accumulation of application layers. The film further comprises a porous bioadhesive surface layer (103). Optionally, a stencil layer is present to depict an image, graphic, and/or font, such as those seen in common body art (104). Next, one or more bioactive agent(s) is/are encapsulated within a carrier that is crosslinked with a crosslinking agent and body ink. The drug-carrier-ink crosslinked compound is prepared in a saturated layer and/or placed within in a reservoir (105). Above the ink layer is a protective water resistant layer (106), followed by a soluble slip coating layer (107). On top of the slip coating is a releasably applied substrate (108) that releases upon solvent exposure, allows underlying layers to transfer to the skin or mucosal membrane during application. The solvent may be water, buffered water, saline, buffered saline, and/or a separately included solution. Additionally, washing the skin with warm water, applying a solution, and/or massaging the application area prior to, and/or during application may be suggested. An application to the skin, may be applied to the inside of the wrist (109), and leave visible semi-permanent body ink on the skin or mucosal membranes once removed (110). Removal of the application film may be accomplished by peeling it off, rubbing with an oil or cream, and/or using gentle exfoliation. A penetration enhancing solution may be separately included for use prior to application.

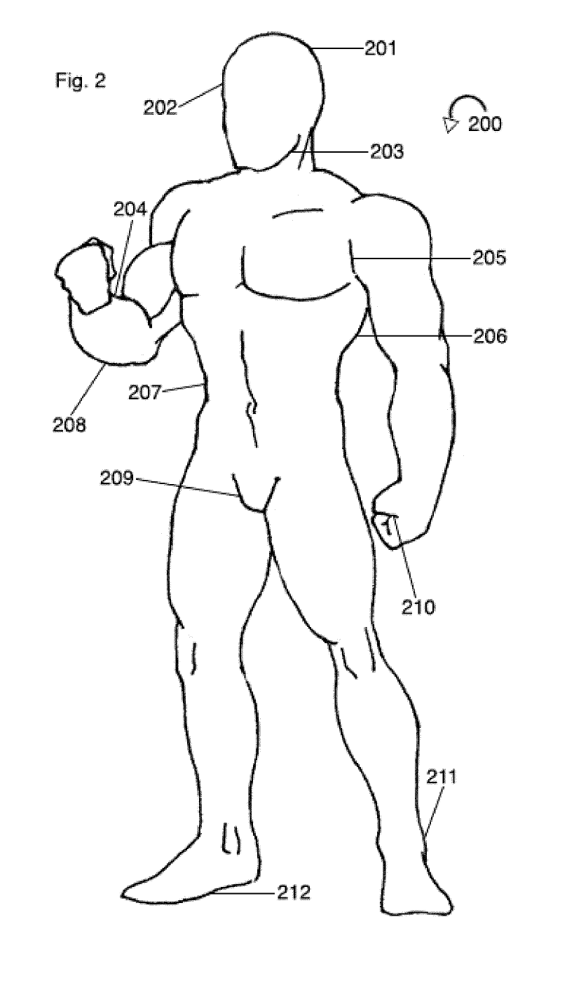

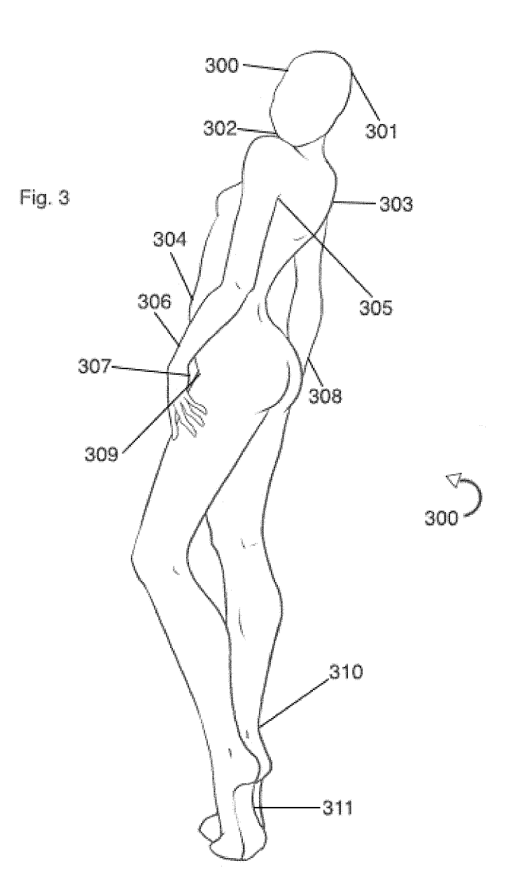

Prime placement, such as displayed in FIG. 2 on a man and FIG. 3 on a woman, includes but is not limited to the scrotum (209), the intimate area (309), jaw (203 and 302), forehead (202 and 300), armpit (205 and 305), scalp (201 and 301), back (206 and 303), abdomen (207 and 304), forearm—dorsal side (208 and 308), forearm—ventral side (204 and 306), palm of hand (210 and 307), ankle (211 and 310), and the arch of foot (212 and 311). Prime placement is optionally recommended when applying a composition to the skin. Not depicted, but additionally included, are applications to mucosal membranes which provide a faster delivery compared to epidermal applications. Therefore, mucosal applications are considered to be prime. Furthermore, the lungs provide the fastest route for medicament to enter the bloodstream. However, generally the effect is very short term. The current invention provides time release drug delivery by the method described herein including applications intended to be inhaled. Therefore the lungs and all inhaled products are considered to be primly placed.

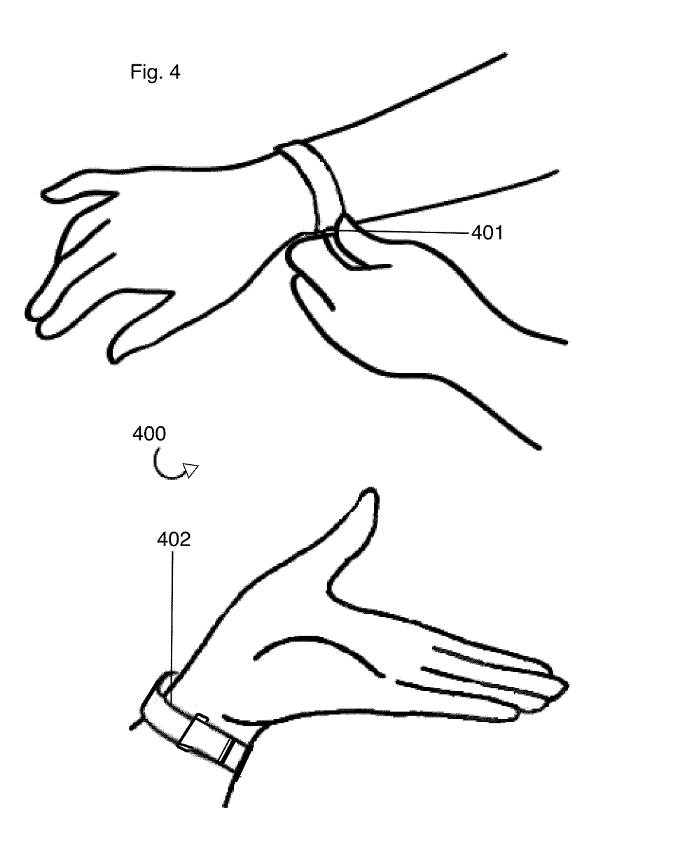

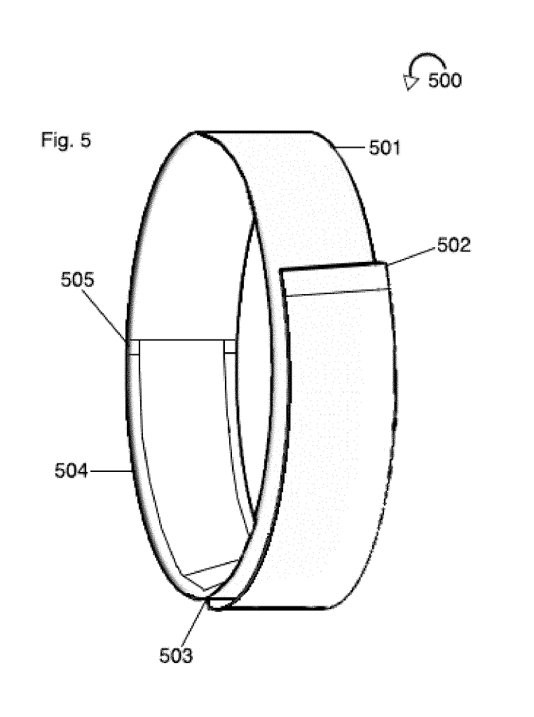

Occlusion products and delivery devices also focus on targeting prime areas of the body to achieve superior penetration such as illustrated in FIG. 4 and FIG. 5. The example depicted in FIG. 4 shows a bracelet or band that can be worn on the wrist (401). On the interior of the band, the drug-carrier crosslinked preparation fits securely against the skin (402). FIG. 5 further illustrates a close up of the band (501) showing the interior where the drug-carrier crosslinked agent is a film contained within the band (504). A rim attached to the band holds the preparation in place on the inside of the band (505). Optionally, the film has a semi-firm yet flexible backing that can be inserted into the interior rim of the band in a refillable application. Optionally, the preparation adheres to the inside of a band with a sticky backing. If the delivery devise is not refillable than it is disposable. A disposable devise may be designed for one or more uses.

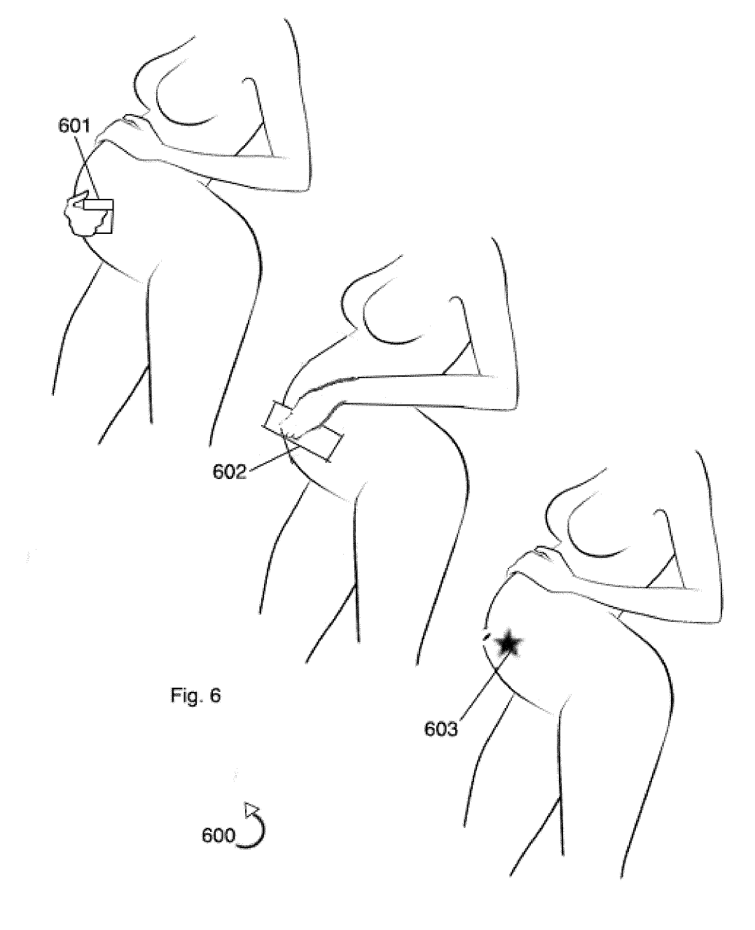

The inventive composition, illustrated in FIG. 6, shows a step by step process for applying a semi-permanent tattoo. The method requires the skin to be waxed, shaved, or essentially free of long hairs prior to application. In this example bioactive agent(s) is/are specific for pregnancy, and may be self applied by a pregnant women according to the process of cleansing the skin with warm water, optionally applying a penetration enhancing solution, and putting the drug delivery application face down on the skin (601). Next, a saturated cloth or a moist towelette comprising a solvent, such as water, is applied over the application (602), releasing the substrate and transferring the application to the skin (603). It is preferred that the solvent be warm water. Once the transfer is complete, the tattoo ink continues to darken for a period of 12-48 hours. The application film is optionally removable after the necessary amount of time has occurred for transfer to skin or mucosal membranes to be completed. The transfer time depends on various components of the application, but generally ranges between 60 seconds to one hour. After removal of the application film, the body ink is systemically bioactive until the ink vanishes from the application point, at which time the user can reapply.

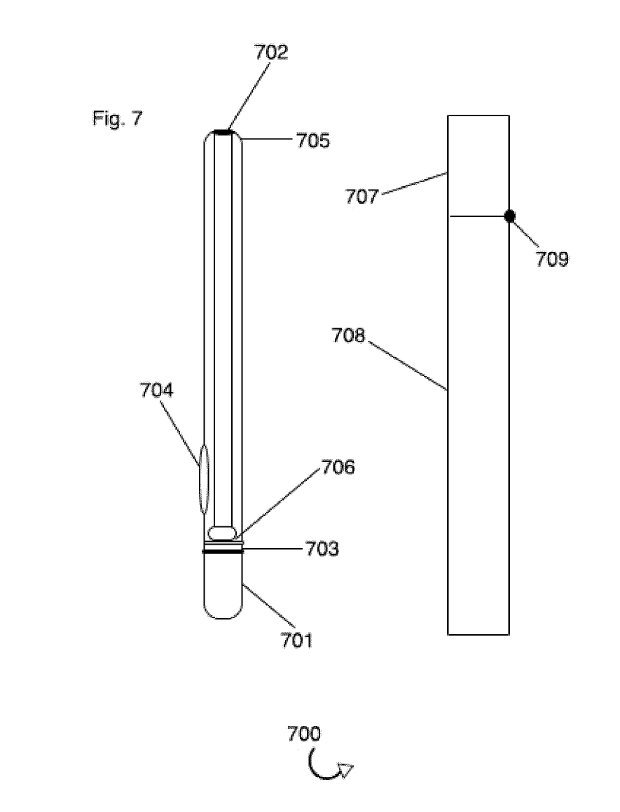

In FIG. 7. bioactive agents formulated for inhalation may be disbursed from an inhalation devise such as a vaporizer, humidifier, e-cigarette, nasal inhaler, oral inhaler, pipe, cigar, and/or cigarette. For example, there is optionally a porous tip to insert in the mouth or nose (701), a reservoir that comprises bioactive agent(s) (702), an optional solar, and/or battery system (703), an optional off/on button or switch (704), a chamber that holds the reservoir and connects to the soft tip (705), a heating unit (706). Furthermore the product optionally includes a protective storage case. A protective storage case optionally has a top (707), a base (708) and a hinge to open and close the top (709). An inventive composition, comprising one or more bioactive agents is/are optionally prepared for inhalation to the lungs of a mammal in a spray, a mist, a liquid, a powder, a dried plant, a syrup, a wax, an e-liquid, an oil, a vapor, a bioactive ink and/or a dye.

1. Drugs, herbs, and/or other bioactive agents, encapsulated in a carrier that is crosslinked with a crosslinking agent for non-invasive drug delivery applications that work locally, systemically, and/or provide a sensory experience for a predetermined amount of time.

2. The inventive composition of claim 1 wherein bioactive agent(s) is/are delivered with synthetic and/or organic carriers comprising one or more from the group of peptides, agar agar, arginine, aloe vera, liposomes, polymers, disc shaped particles, sphere shaped particles, nanoparticles, micro-particles, gold nanoparticles, silver nanoparticles, copper nanoparticles, multistage vectors, diatomaceous earth, moroccan red clay, pollen, and/or biologically acceptable organic moieties.

3. The inventive composition of claim 1 where crosslinking agent(s) is/are selected from genipin (GNP) and/or proanthocyanidin (PA).

4. The inventive composition of claim 1 prepared at a pH of 7 to 7.5.

5. The inventive composition of claim 1 comprising an organic solvent selected from water, saline, buffered saline, buffered water, vinegar, acetic acid, and/or isopropyl alcohol.

6. The inventive composition of claim 1 additionally comprising a scent and/or flavoring.

7. The inventive composition of claim 1 prepared with a thickening agent such as natural gums, polysaccharides, and/or xanthan gum.

8. The inventive composition of claim 1 additionally comprising an oil and/or butter selected from the group comprising coconut, jojoba, cocoa, almond, cashew, shea, olive, sunflower, avocado, argan, flaxseed, and/or castor oil.

9. The inventive composition of claim 1 additionally comprising an emollient such as a wax selected from the group of beeswax, bayberry wax, candelilla wax, carnauba wax, and/or castor wax.

10. The inventive composition of claim 1 wherein the bioactive agent(s) comprise one or more from the group of marine ingredients, minerals, botanicals, peptides, mucco-polysaccharides, polyphenols, trans-resveratrol, minerals, trace minerals, vitamins, trace elements, antioxidants, fruit acids, fungi, activated charcoal, diatomaceous earth, exogenous ketones, ketone esters, ketone salts, fermentations, distillations, probiotics, amino acids, amino acid salts, fatty acids, Co-Q10, omega-3, omega-6, proteins, essential oils, herbs, nutrients, whole foods, organic whole foods, sprouted foods, raw foods, superfoods, and/or other bioactive agents in the form of extracts, derivatives, and/or concentrates.

11. The inventive composition of claim 1 in the form of a spray, a mist, a liquid, a powder, a syrup, a wax, an oil, a vapor, a film, a foam, a cream, a lotion, a gel, a jelly, a butter, a moose, a scrub, a soak, a paste, a serum, a dye, an e-liquid, and/or a solid.

12. The inventive composition of claim 1 worn on or in a product and/or delivery devise such as a(n) bracelet, wristband, wrap, tie, sticker, temporary tattoo, band aid, impregnated tampon, condom, vaginal ring, suppository, penile band, sponge, contact lens, inhaler, cigarette, e-cigarette, cosmetic product, corset, headband, eye pillow, bodysuit, undergarment, diaper, contact lens, e-cigarette, inhaler, vaporizer, humidifier, neoprene wrap, pet collar, controlled delivery device and/or device wherein the components are intended to be discarded after being used.

13. The inventive composition of claim 1 as a treatment, prevention, or remedy such as a(n) immune system booster, hangover prevention, hangover treatment, pain reliever, sleep aid, weight loss supplement, sunscreen, full spectrum UV radiation protection, addiction recovery supplement, STD prevention, male contraceptive, female contraceptive, systemic insect repellent, mosquito repellent, tick repellent, flea repellent, sleep aid, vitamin, multi-vitamin, women's multi-vitamin, men's multivitamin, prenatal vitamin, breastfeeding vitamin, children's vitamin, infant vitamin, cognitive function booster, concentration enhancer, detoxifier, digestive aid, metabolism booster, energizing supplement, vitamin B complex, analgesic, anti-bacterial, antibiotic, probiotics, plant based recovery supplement, workout booster, brain enhancing supplement, study and concentration help, mental focus and memory supplement, heart health supplement, lung recovery supplement, plant calcium supplement, plant protein supplement, raw organic protein supplement, collagen builder, fermented food supplement, probiotic supplement, essential fatty acid supplement, Co-Q10 supplement, omega-3 supplement, omega-6 supplement, fatty acid supplement, enzyme supplement, mineral supplement, trace mineral supplement, amino acid supplement, antiviral, antioxidant complex, anti-inflammatory, polyphenol supplement, peptide supplement, organic raw food supplement, sprouted food supplement, exogenous ketone supplement, ketone esters, superfood supplement, herbal defense, virus protection, virus recovery, anti-fungal, anti-cholinergic, sexual enhancer, aphrodisiac, herbal mood enhancer, blood born illnesses protection, cancer prevention/treatment, heart disease prevention/treatment, Alzheimer's prevention/treatment, dementia prevention/treatment, high blood pressure prevention/treatment, high cholesterol prevention/treatment, cardiovascular disease prevention/treatment, diabetes prevention/treatment, migraine prevention/treatment, hair loss prevention/treatment, colic (babies) prevention/treatment, intestinal parasite prevention/treatment, ulcer prevention/treatment, arthritis prevention/treatment, acne prevention/treatment, eczema prevention/treatment, fungal infection prevention/treatment, energy supplement, depression prevention/treatment, dermatitis prevention/treatment, epileptic seizure prevention/treatment, gallbladder stone prevention/treatment, prostate protection, kidney stone prevention/treatment, hemorrhoid prevention/treatment, stretch mark prevention/treatment, cellulite prevention/treatment, varicose veins prevention/treatment, beautifying treatment/therapy, visible aging prevention and/or physical aging prevention, and/or combination thereof, and/or similar therapy, treatment, beauty routine, cosmetic product, preventative treatment, cure, and/or remedy.

14. The inventive composition of claim 1 additionally comprising one or more body inks selected from the group comprising genipin, henna, woad, madder, achiote, indigo, gold, copper, silver, onion, blueberries, coffee, mango, papaya, cranberry, chlorophyll, beetroot, barberry, raspberry, paprika, saffron, safflower, birch, tea, bay leaves, turmeric, carrot, cocoa, nettles, goldenrod, lily of the valley, hibiscus, roses, cherries, walnut, black walnut, espresso, dandelion, oak bark, sunflower, St. John's wort, and/or citrus dyes as described in various embodiments herein.

15. The ink inclusive composition of claim 14 prepared for application as a temporary tattoo comprising a porous bioadhesive surface, a bioactive drug-carrier-crosslinking agent compound, a body ink component, a protective water resistant layer, a soluble slip coating layer, and a releasably applied substrate.

16. The ink inclusive composition of claim 14 prepared for application as semi-permanent body ink.

17. The ink inclusive composition of claim 14 prepared for application as a hair dye and/or cosmetic product.

18. The ink inclusive composition of claim 14 prepared for application as a self tanning product.

19. The ink inclusive composition of claim 14 for which the visual disappearance of ink signifies the end of the dosage.
